(19) United States
(12) Patent Application Publication
(10) Pub. No.: US 2024/0277230 A1
(43) Pub. Date: Aug. 22, 2024

(54) OPTICAL MEDICAL EXAMINATION DEVICE

(71) Applicant: HEINE Optotechnik GmbH & Co. KG, Gilching (DE)

(72) Inventors: Roman RAAB, Gilching (DE); Felix Michel, Gilching (DE)

(21) Appl. No.: 18/442,581

(22) Filed: Feb. 15, 2024

(30) Foreign Application Priority Data
Feb. 16, 2023 (DE) 10 2023 103 906.7

(51) Int. Cl. A61B 5/00; G02B 27/28; H05B 47/155; H05B 47/17

(52) U.S. Cl. CPC A61B 5/0077; A61B 5/441; G02B 27/288; H05B 47/155; H05B 47/17

(57) ABSTRACT

An examination device for optical medical examinations having an optics system and an illumination device. The illumination device includes at least one first light source and at least one second light source arranged in such a way that they illuminate an examination region together. The first light source is configured to emit white light and the second light source is configured to emit light that is bluer than the white light. The light emitted by the at least one second light source has a wavelength in the range of 400 nm to 550 nm and/or the illumination device includes multiple first light sources and multiple second light sources, the first light sources and the second light sources are arranged adjacent to each other and form pairs, wherein the illumination device includes at least one polarization filter that covers every second pair.

### FIELD OF THE DISCLOSURE

The disclosure relates to an examination device for optical medical examinations, in particular a dermatoscope.

### BACKGROUND

Optical examination devices, in particular dermatoscopes, are known and usually comprise an optics system as well as an illumination device, with which the examination region U can be illuminated.

In particular when examining the skin, it is necessary that the examination region can be illuminated with light comprising different colour temperatures and different polarizations as various structures of the skin are best visible in different lighting conditions.

However, at the same time, the examination devices must be particularly compact to ensure good operability.

There is a need to provide an examination device for optical medical examinations that can provide light comprising different colour temperatures and in doing so is particularly compact.

### SUMMARY

There is provided an examination device for optical medical examinations, in particular a dermatoscope, comprising an optics system and an illumination device. The optics system has an optical axis and defines the examination region. The illumination device comprises at least one first light source and at least one second light source, wherein said at least one first light source and said at least one second light source are arranged in such a way that they illuminate the examination region together. Said at least one first light source is configured to emit white light and said at least one second light source is configured to emit light that is bluer than the white light emitted by said at least one first light source. The light emitted by said at least one second light source has a wavelength in the range of 400 nm to 550 nm and/or the illumination device comprises multiple first light sources and multiple second light sources. Each one of the first light sources and one of the second light sources are arranged adjacent to each other and form a pair, wherein the pairs have at least one pair as a first pair and at least another pair as a second pair, wherein the illumination device comprises at least one polarization filter that covers said at least one second pair.

The inventors have recognised that it is not necessary to use multi-chromatic light sources, for example multi-coloured LEDs, in order to provide light with different colour temperatures, but rather the required sufficient changes to the colour temperature can already be achieved by means of two light sources which emit light with different spectra. As a result, the light sources can be designed considerably more compact because, for example, single-coloured LEDs are considerably smaller than multi-coloured LEDs.

The term “bluer” is to be understood, for example, in terms of colour and colour perception and not in terms of a lower wavelength of the emitted light. “Bluer” can mean in particular a higher colour temperature.

The terms “multi-coloured” and “single-coloured” do not relate to the wavelength emitted at a point in time, but rather the possibility of the light source to change the colour and colour temperature of the emitted light or the absence of such a possibility.

“Illuminating” is understood to mean within the scope of this disclosure that the cone of light of said at least one first light source and the cone of light of said at least one second light source overlap in the examination region, in particular in the majority of the examination region, more particularly in the entire examination region.

The light sources are LEDs, for example.

The examination region can be a region about the optical axis with the diameter of the optics system.

In an embodiment of the disclosure, the white light emitted by said at least one first light source has a colour temperature between 4000 K and 6000 K, in particular between 4500 K and 5500 K, more particularly at 5000 K or has a colour temperature between 2500 K and 4500 K, in particular between 3000 K and 4000 K, more particularly at 3500 K; and/or the bluer light emitted by said at least one second light source has a wavelength in the range of 450 nm to 500 nm, in particular at 475 nm, or a colour temperature between 5500 K and 7500 K, more particularly between 6000 K and 7000 K, in particular at 6500 K. In this way, the optimal lighting for different examinations is provided.

The aspect, wherein the bluer light emitted by said at least one second light source has a colour temperature between 5500 K and 7500 K, more particularly between 6000 K and 7000 K, in particular at 6500 K, is suitable for the purpose and it is independent aspect, in particular from the aspect that the light emitted by said at least one second light source has a wavelength in the range of 400 nm to 550 nm.

To enable central operation, the examination device can comprise a control unit that controls said at least one first light source and said at least one second light source.

In an embodiment, the examination device, in particular the control unit, is configured in such a way that said at least one first light source is switched on when the illumination device is activated, thereby determining an initial colour temperature. The user, for example, can become accustomed to this.

Said at least one first light source is always switched on, for example, or switched on at least for the majority of the different illumination modes.

In an embodiment, the examination device, in particular the control unit, is configured in such a way that the colour temperature of the light emitted by the illumination device can be set in a colour temperature range, wherein the lower limit of the colour temperature range is between 4000 K and 6500 K, in particular between 4500 K and 6000 K, more particularly at 5000 K, and/or the upper limit of the colour temperature range is between 10000 K and 14000 K, in particular between 11000 K and 13000 K, more particularly at 12000 K. Optical examinations are optimally possible in these colour temperature ranges.

To reduce the colour temperature in a simple way, the examination device, in particular the control unit, can be configured in such a way that said at least one second light source can be switched on in addition to said at least one first light source.

In particular, said at least one second light source is always only switched on in addition to said at least one first light source.

In an embodiment, the examination device, in particular the control unit, is configured in such a way that the luminous intensity of the light emitted by said at least one second light source is changeable, in particular in multiple discrete levels. As a result, the colour temperature can be set simply and in the case of discrete levels also repeatably.

For example, the luminous intensity of the light emitted by said at least one first light source is also changeable, in particular in multiple discrete levels. As a result, the brightness can be set simply and in the case of discrete levels also repeatably.

In particular, the light emitted by the first light sources always has a higher luminous intensity than that of the second light sources.

In an embodiment, the first light sources and the second light sources are arranged about the optical axis of the optics system.

In particular, the number of first light sources is the same as the number of second light sources.

The arrangement about the optical axis is, for example, annular.

The LEDs are inclined away from the optical axis, for example, at an angle ranging from 35° to 55°, in particular an angle of 45°.

In an embodiment, in each case, one of the first light sources and one of the second light sources are arranged adjacent to each other and form a pair.

The pairs can have the same spacing to each other in the circumferential direction.

In an embodiment of the disclosure, the pairs have at least one pair as a first pair and at least another pair as a second pair, wherein the illumination device comprises at least one polarization filter that covers said at least one second pair.

The polarization filter of the illumination device is in particular a linear polarization filter.

For example, multiple first and second pairs are provided, wherein all second pairs are covered by the polarization filter of the illumination device.

Within the scope of this disclosure, “covered” means in particular that the polarization filter is located in the cone of light of the first and second light sources of the pair.

The polarization filter of the illumination device is located in particular between the first and second light sources and the examination region.

To increase the contrast of certain structures to be examined, said at least one polarization filter of the illumination device can polarize passing light linearly, for example, in a first polarization direction, and/or the optics system comprises a polarization filter that polarizes the passing light linearly in a second polarization direction, in particular wherein the second polarization direction is perpendicular to the first polarization direction so that the polarization filter of the optics system blocks light with a polarization in the first polarization direction.

For particularly uniform illumination, the first pairs and the second pairs can be located alternately in the circumferential direction.

In an embodiment, the illumination device comprises several separate polarization filters, wherein one of the polarization filters covers in each case one of the second pairs.

The separate polarization filters polarize passing light all in the same first polarization direction.

In an embodiment, the examination device, in particular the control unit, is configured in such a way that either the first light sources and the second light sources of said at least one first pair or the first light sources and the second light sources of said at least one second pair are switched on when the illumination device is activated, thereby providing either polarized or non-polarized light.

For example, light sources of a first pair and a second pair are never switched on simultaneously.

In particular, the light sources of all first pairs or all second pairs are always switched on if the illumination device is activated.

In an embodiment of the disclosure, the examination device, in particular the control unit, is configured in such a way that when the light sources of said at least one first pair are switched off in order to activate the light sources of said at least one second pair, the light sources of said at least one second pair are switched on in such a way that the luminous intensities of the light emitted by the light sources of the second pair correspond to the luminous intensities of the light that was emitted last by the corresponding light source of said at least one first pair, and/or vice versa. In this way, it ensures that only the polarization of the light changes in the examination region so that the user is not required to undertake any further settings.

For example, the optics system is a magnifying optics system comprising an imaging sensor and/or a camera in order to simply the examination.

### BRIEF DESCRIPTION OF THE DRAWINGS

Additional features and advantages of the disclosure are found in the following description as well as the attached drawings to which reference is made. In the drawings:

FIG. 1 shows an examination device according to an embodiment of the disclosure in a perspective view,

FIG. 2 shows a schematic section view through a part of the examination device according to FIG. 1, and

FIG. 3 shows a conceptional view of the illumination device and the optics system in an underside view of the examination device according to FIG. 1.

### DETAILED DESCRIPTION

Lists having a plurality of alternatives connected by “and/or”, for example “A, B and/or C” are to be understood to disclose an arbitrary combination of the alternatives, i.e. the lists are to be read as “A and/or B and/or C” or as “at least one of A, B or C”. The same holds true for listings with more than three items.

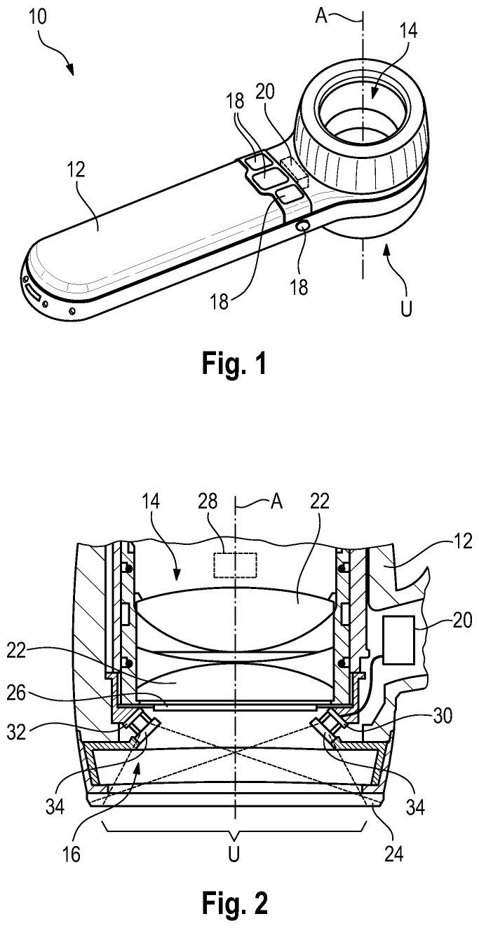

In FIG. 1, an examination device 10 for optical medical examinations is shown.

The examination device 10 is a dermatoscope, for example, and is designed as a handheld device in the shown embodiment.

The examination device 10 comprises a housing 12, an optics system 14, an illumination device 16 (FIG. 2), several push buttons 18 and a control unit 20.

The housing 12 has a grip region and a head region, wherein the optics system 14 and the illumination device 16 are located in the head region.

The push buttons 18 are provided on the grip region, in particular on the end of the grip region facing the head region.

The push buttons 18 are located, for example, in such a way that they can be operated using the thumb when the grip region is gripped using the hand.

The optics system 14, the illumination device 16 and the control unit 20 are located within the housing 12. In addition, an energy storage device, such as an accumulator or a battery, can be provided within the housing 12, in particular within the grip region.

FIG. 2 shows a schematic section through the head region of the housing 12 as well as the optics system 14 and the illumination device 16 situated in it.

The housing 12 has a channel in the head region, in said channel the optics system 14 is located.

The optics system 14 comprises several lenses 22, a cover glass 24 on the patient side, said cover glass 24 closing the channel on the patient side, and a polarization filter 26.

It is also conceivable that the optics system 14 has a camera 28 (indicated by dashed lines). An imaging sensor is also conceivable, for example, instead of the camera.

The optics system 14 comprises an optical axis A that runs centrally through the lenses 22.

The optics system 14 covers a region directly in front of the cover glass 24 that represents the examination region U.

The examination region U extends about the optical axis A and has approximately the diameter of the optics system 14, i.e. the lenses 22.

The lenses 22 are designed in such a way that the image of the examination region U is shown magnified for a user on the side facing away from the examination region U.

The polarization filter 26 is located, for example, between the lenses 22 and the cover glass 24.

The illumination device 16 is also provided in the head of the housing 12.

The illumination device 16 extends about the optical axis A and is located between the cover glass 24 and the lenses 22.

The illumination device 16 comprises multiple first light sources 30, multiple second light sources 32 as well as several polarization filters 34.

The light sources 30, 32 are, for example, light-emitting diodes (LEDs).

The light sources 30, 32 are located around the optical axis A and form an angle between 35° and 55°, in particular 45° relative to the optical axis A.

The polarization filters 34 are located in front of the light sources 30, 32, i.e. towards the examination region U.

In the shown embodiment, the polarization filters 34 are also arranged at an angle in the same angle as the light sources 30, 32 relative to the optical axis A.

The light sources 30, 32 are connected electrically to the control unit 20, wherein the control unit 20 is configured to control the first light sources 30 and the second light sources 32, in particular separately from each other or in groups, as will be explained below.

The required energy for this is provided by the energy storage device or a cable connected to a power source.

The first light sources 30 and the second light sources 32 emit light each in a cone of light (indicated by dashed lines), wherein the cone of light of the individual light sources 30, 32 overlap in the examination region U. Thus, the first light sources 30 and the second light sources 32 illuminate the examination region U together.

In particular, at least the majority of the examination region U, more particularly however the entire examination region U, is illuminated by the light sources 30, 32 together.

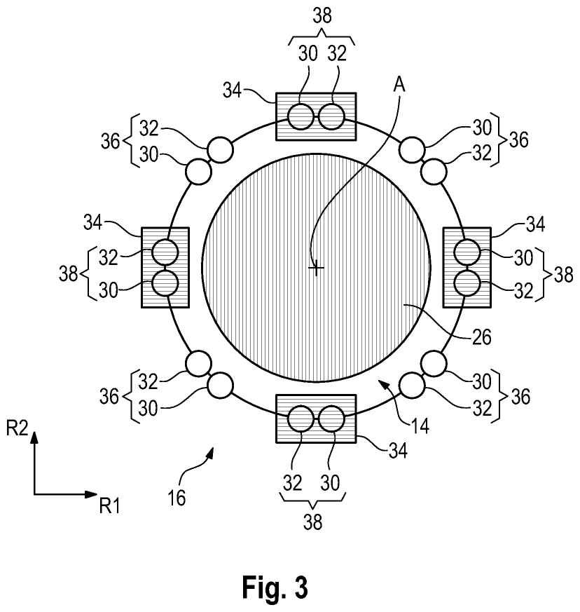

In FIG. 3, a view of the optics system 14 and the illumination device 16 is shown conceptionally from underneath, i.e. through the cover glass 24.

It is clearly evident that the optics system 14, of which only the polarization filter 26 can be seen, is designed circularly and extends the first light sources 30 and the second light sources 32 along the circumference of the optics system 14.

The first light sources 30 and the second light sources 32 are located in the circumferential direction about the optical axis A, circularly in the shown embodiment.

The same number of first light sources 30 and second light sources 32 are provided, in the shown embodiment eight of each.

The first light sources 30 emit white light, for example with a colour temperature between 4000 K and 6000 K, in particular between 4500 K and 5500 K. For example, the first light sources 30 emit white light with a colour temperature of 5000 K.

The second light sources 32 emit blue light, for example with a wavelength ranging from 400 nm to 550 nm, in particular from 450 nm to 500 nm, more particularly at 475 nm.

It is also conceivable that the first light sources 30 emits light with a colour temperature between 2500 K and 4500 K, in particular between 3000 K and 4000 K, for example of 3500 K, and the second light sources emits light with a colour temperature between 5500 K and 7500 K, in particular between 6000 K and 7000 K, for example at 6500 K.

The first light sources 30 and the second light sources 32 are arranged alternately in the circumferential direction about the optical axis A, wherein each first light source 30 forms a pair with a directly adjacent second light source 32.

The pairs of light sources 30, 32 are spaced apart at the same spacing in the circumferential direction. For example, the spacing between the light sources 30, 32 of a pair is less than the spacing between the two adjacent pairs.

The polarization filters 34 of the illumination device 16 are provided for half of the pairs of light sources 30, 32. The polarization filters 34 are parts that are separate from each other.

In the shown embodiment, four polarization filters 34 are provided, wherein each of these polarization filters 34 cover the light sources 30, 32 of one of the pairs.

This means that the cone of light of the light sources 30, 34 of this pair runs through the corresponding polarization filter 34 so that the light originating from these light sources 30, 32 is polarized in the examination region U accordingly.

As a result of the polarization filters 34, the pairs differ from each other, wherein in the following, solely for differentiation purposes, the pairs of light sources 30, 32 without any corresponding polarization filters are termed the first pairs 36 and the pairs of light sources 30, 32 covered by a polarization filter 34 are termed the second pairs 38.

The first pairs 36 and the second pairs 38 are located alternately in the circumferential direction about the optical axis A.

It is conceivable that only a polarization filter 34 that covers all second pairs 36 is provided for the second pairs 36.

The polarization filters 34 have a first polarization direction R1, wherein the light passing through the polarization filter 34 is polarized linearly in the first polarization direction R1.

The polarization direction R1 is indicated in FIG. 3 by means of the hatching.

The polarization filter 26 of the optics system 14 is similarly a linear polarization filter with a second polarization direction R2. The polarization filter 26 polarizes the light passing through it linearly in the direction of the second polarization direction R2.

The second polarization direction R2 is perpendicular to the first polarization direction R1 and is indicated by the hatching of the polarization filter 26. The polarization filter 26 of the optics system 14 thus blocks light with a polarization in the first polarization direction R1, in particular reflections of the light sources 30, 32 of the second pairs 36. Thus, the polarization filters 34 of the illumination device 16 can be regarded as polarizers and the polarization filter 26 of the optics system 14 as an analyzer.

The illumination device 16 can be activated and deactivated during the examination using the examination device 10, in particular by means of one of the push buttons 18.

The light sources 30, 32 are controlled by means of the control unit 20 depending on the actuation of the push buttons 18.

By means of the push buttons 18, various illumination modes can be selected, in which the first light sources 30 and the second light sources 32 of the various pairs 36, 38 are switched on or off and emit light with different luminous intensities.

As soon as the illumination device 16 is activated, at least the first light sources 30 of the first pairs 36 or the first light sources 30 of the second pairs 38 are switched on.

In each of the illumination modes, either the first light sources 30 and, if applicable, the second light sources 32 of the first pairs 36 are switched on and the light sources 30, 32 of the second pairs 38 are switched off, or the light sources 30, 32 of the first pairs 36 are switched off and the first light sources 30 and, if applicable, the second light sources 32 of the second pairs 38 are switched on.

It is also conceivable that the first light sources 30 of the first pairs 36 or the first light sources 30 of the second pairs 38 are not always switched on, but at least for more than half of the various illumination modes.

The luminous intensity of the light emitted by the first light sources 30 can be changed. For example, by actuating one of the push buttons 18, discrete levels of different luminous intensities can be switched through and selected in order to change the brightness in the examination region U.

In particular, the first light sources 30 are always switched on when the illumination device 16 is activated, either the first light sources 30 of the first pairs 36 or those of the second pairs 38.

The second light sources 32 of the corresponding pairs can be activated in addition to the switched-on first light sources 30 of the first and second pairs 36, 38. This can also be executed by actuating one of the push buttons 18.

In doing so, the luminous intensity of the light emitted by the second light sources 32 is changeable. For example, the luminous intensity is changeable via multiple discrete levels, which can be selected or switched through by means of one of the push buttons 18.

For example, the light emitted by the first light sources 30 always has a higher luminous intensity than the light emitted by the second light sources 32.

By additionally switching on the second light sources 32, the colour temperature of the light changes in the examination region U as the examination region U is illuminated by the light of the first and the second light sources 30, 32.

By switching on the second light sources 32, the colour temperature of the light in the examination region U shifts to blue, thereby making certain structures in the object to be examined, in particular the skin, more visible.

If the luminous intensity of the light of the second light sources 32 is subsequently increased, the colour temperature of the light in the examination region U shifts further to blue.

For example, the lower limit of the colour temperature range that is attained in this way and permitted by the control unit 20 is between 4000 K and 6500 K, in particular between 4500 K and 6000 K, more particularly at 5000 K, and/or the upper limit of this colour temperature range is between 10000 K and 14000 K, in particular between 11000 K and 13000 K, more particularly at 12000 K.

The polarization of the light in the examination region U can take place by switching between the pairs 36, 38 of light sources 30, 32, thus by switching off the light sources 30, 32 of one of the pairs 36, 38 and by switching on the light sources 30, 32 of the other pairs 38, 36.

The switching between the light sources 30, 32 of the first pairs 36 and the second pairs 38 can be triggered by actuating one of the push buttons 18.

If only the light sources 30, 32 of the first pairs 36 are switched on, the light in the examination region U is thus non-polarized. However, if the light sources 30, 32 of the second pairs 38 are used, the light in the examination region U is polarized along the first polarization direction R1.

To make changing from non-polarized light to polarized light in the examination region U and vice versa as convenient as possible for the user of the examination device 10, the first light sources 30 and the second light sources 32 of the pairs 36, 38 switched on are operated like the light sources 30, 32 of the pairs 36, 38 switched off have been operated until then, in particular in relation to luminous intensities.

If, for example, there is a change from non-polarized light (i.e. the first pairs 36) to polarized light (i.e. the second pairs 38), the luminous intensities of the light of the first light sources 30 and the second light sources 32 of the second pairs 38 are set in such a way that they correspond to the most recently used luminous intensities of the light of the first light sources 30 or the second light sources 32 of the first pairs 36.

To switch from polarized light to non-polarized light, the opposite applies accordingly.

By means of the examination device 10, the colour temperature and also the polarization of the light in the examination region U can thus be set without using multi-coloured LEDs for this.

In this way, a compact, but economical examination device 10 is attained.

1. An examination device for optical medical examinations, comprising an optics system and an illumination device,
wherein the optics system has an optical axis and defines an examination region,
wherein the illumination device comprises at least one first light source and at least one second light source, wherein said at least one first light source and said at least one second light source are arranged in such a way that they illuminate the examination region together, and
wherein said at least one first light source is configured to emit white light and said at least one second light source is configured to emit light that is bluer than the white light emitted by said at least one first light source,
and at least one of wherein the light emitted by said at least one second light source has a wavelength in a range of 400 nm to 550 nm; or wherein the illumination device comprises multiple first light sources and multiple second light sources, wherein in each case one of the first light sources and one of the second light sources are arranged adjacent to each other and form a pair, wherein the pairs have at least one pair as a first pair and at least another pair as a second pair, wherein the illumination device comprises at least one polarization filter that covers said at least one second pair.

2. The examination device according to claim 1, wherein the white light emitted by said at least one first light source has a colour temperature between 4000 K and 6000 K, or has a colour temperature between 2500 K and 4500 K.

3. The examination device according to claim 1, wherein the light emitted by said at least one second light source has a wavelength in the range of 450 nm to 500 nm, or has a colour temperature between 5500 K and 7500 K.

4. The examination device according to claim 1, wherein the examination device comprises a control unit that controls said at least one first light source and said at least one second light source.

5. The examination device according to claim 1, wherein the examination device is configured in such a way that said at least one first light source is switched on when the illumination device is activated.

6. The examination device according to claim 1, wherein the examination device is configured in such a way that a colour temperature of the light emitted by the illumination device is settable in a colour temperature range, wherein at least one of a lower limit of the colour temperature range is between 4000 K and 6500 K or a upper limit of the colour temperature range is between 10000 K and 14000 K.

7. The examination device according to claim 1, wherein the examination device is configured in such a way that said at least one second light source is only be switched on in addition to said at least one first light source.

8. The examination device according to claim 7, wherein the examination device is configured in such a way that a luminous intensity of the light emitted by said at least one second light source is changeable.

9. The examination device according to claim 8, wherein the examination device is configured in such a way that the luminous intensity of the light emitted by said at least one second light source is changeable in multiple discrete levels.

10. The examination device according to claim 1, wherein the first light sources and the second light sources are arranged about the optical axis of the optics system.

11. The examination device according to claim 1, wherein said at least one polarization filter of the illumination device polarizes passing light linearly in a first polarization direction.

12. The examination device according to claim 11, wherein the optics system comprises a polarization filter that polarizes passing light linearly in a second polarization direction.

13. The examination device according to claim 12, wherein the second polarization direction is perpendicular to the first polarization direction so that the polarization filter of the optics system blocks light with a polarization in the first polarization direction.

14. The examination device according to claim 1, wherein the first pairs and the second pairs are located alternately in a circumferential direction.

15. The examination device according to claim 1, wherein the illumination device comprises several separate polarization filters, wherein each one of the polarization filters of the illumination device covers one of the second pairs.

16. The examination device according to claim 1, wherein the examination device is configured in such a way that either the first light sources and the second light sources of said at least one first pair or the first light sources and the second light sources of said at least one second pair are switched on when the illumination device is activated.

17. The examination device according to claim 16, wherein the examination device is configured in such a way that when the light sources of said at least one first pair are switched off to activate the light sources of said at least one second pair, the light sources of said at least one second pair are switched on in such a way that luminous intensities of the light emitted by the light sources of the second pair corresponds to luminous intensities of the light that was emitted last by the corresponding light source of said at least one first pair, or vice versa.

18. The examination device according to claim 1, wherein the optics system is at least one of a magnifying optics system, comprising an imaging sensor or comprising a camera.

19. The examination device according to claim 1, wherein the examination device is a dermatoscope.
